(19) United States
(12) Patent Application Publication
(10) Pub. No.: US 2012/0324635 A1
(43) Pub. Date: Dec. 27, 2012

(54) Goggles with ear warming means

(76) Inventor: Samuel R. Shapiro, Monroeville, PA (US)

(21) Appl. No.: 13/134,936

(22) Filed: Jun. 21, 2011

(51) Int. Cl. A41D 13/05; A61F 9/02

(52) U.S. Cl. 2/423

(57) ABSTRACT

An ear protection device made of thermally insulating fabric for fastening to goggles for winter sport use. The device includes two downward extensions of fabric disposed to overlie the wearer's ears. Each downward extension includes a pocketed region for holding small self-heating chemical or electrical packs. An additional security pocket may be accessed from an interior headband component to this device.

### CROSS-REFERENCE TO RELATED APPLICATION

This application claims priority to U.S. Provisional Application Ser. No. 61/397,971, filed on Jun. 19, 2010 and entitled “Goggles with Ear Warming Means”, the disclosure of which is fully incorporated by reference herein.

### BACKGROUND OF THE INVENTION

1. Field of the Invention

This invention relates to winter sports goggles and, more particularly, to a novel combination of goggles with adjustable ear warming covers. Preferably, the invention addresses ski goggles having a headband component made as a one piece, foldable ear cover with memory foam. The ear covering includes a plurality of recesses into which can be situated replaceable, external heat packs or electronic heat sources. An additional security pocket may be accessed from inside the headband component.

In winter sports such as skiing, snowboarding, snowmobile riding, bobsledding, and other high-speed outdoor events, it is necessary to shield the eyes of the athlete from wind, precipitation like snow, sleet or rain, and the glare of the sun. Additionally, due to the cold temperature and the added wind chill effect of speed, it is necessary to protect the ears of the athlete from the cold to prevent discomfort and avoid frostbite. Athletes also tend to wear headbands to collect perspiration created by the strenuous activity. In cold weather, a headband further serves to protect the ears of the athlete.

Winter sports goggles have developed to a high degree of technical effectiveness in shielding the eyes from wind and ultraviolet rays with an enclosed frame fitting snugly around the contours of the wearer's nose and face with foam padding around the edges of the goggles. At the same time, such frames include foam-covered openings along their edges to allow moisture to escape and help prevent the goggles from fogging.

2. Relevant Art

Most prior art goggles attach to their wearer's head using an elasticized strap for fitting the goggles and their foam frame edges snugly against the wearer's skin thus preventing airflow into the wearer's eyes. Such goggles are usually pulled on and off over the wearer's head interfering with glasses, headbands, hats and/or other headgear. Once in place, the wearer may find it difficult to get a proper adjustment of his/her elasticized strap especially during vigorous physical activity. Elasticized straps that are relatively narrow may tend to concentrate pressures to a narrow band around the head, thereby increasing the possibility of interfering with the wearer's blood circulation in that region. In addition, the constant tensioning of an elasticized strap over time causes the strap to lose elasticity and become ineffective.

To protect the ears of the winter outdoor athlete, hats pulled down over the ears often prove defective. Sometimes, the ears get only partially covered by a hat that tends to “migrate” upwards and/or forwards. In addition, loosely knit hats are stretchable and allow chilling air to blow through. Earmuffs are often bulky and quite unfashionable with the interconnecting metal band posing another hindrance for wearers to contend with and/or navigate other headgear around.

The traditional headband is a width of stretchable cloth sufficient to cover the wearer's ears. When combined with stand alone, protective eye wear such as ski goggles, yet another round of problems arise. First, each device of the separate head band/eye apparel arrangement competes for space and jockeys for position on the wearer's head. Thus, adjusting one's goggles displaces the headband and vice versa. Second, an ear protector of constant width around the head interferes with one's hairdo and resulting in “ski hair”, “hat head” or “headband hair.” The alternative to the headband is a pull down hat, earmuffs or simply having cold ears.

Schulze U.S. Pat. No. 5,421,037 disclosed a goggles-headband combination in which the goggle-retaining strap is covered with a single length of cloth of a and width to encircle the retaining strap from opposed end edges of the goggles. A blank piece of cloth was sized to completely encircle the retaining strap from opposed side edges of the goggles, and cover the wearer's ears. It employed cooperative releasable fastening means secured to both the longitudinal dimension of strips as well as the latitudinal dimensions of the cloth for releasable securement of the cloth to the retaining strap.

The preceding device had a myriad of drawbacks. First the earpiece was not retained around the ears of the wearer. As the skier/snowboarder gained speed, the wind picked up the material proximate the ears and blew it outward exposing the ears of the wearer. The means for attaching cloth to the retaining strap allowed no adjustment for cinching about the strap. The ear covering itself could twist about causing wearer discomfort and possible further risk of ear exposure to the elements.

Lacore et al. U.S. Pat. No. 5,617,589 described a pair of sporting goggles with a headband that also served as an earmuff. That device incorporated the ear covering into the band itself, not allowing for removal of such covering without removing the entire goggles assemblage.

It would be advantageous to have a device that protects its wearer's ears under the extreme conditions of down hill skiing or snowmobile riding and/or snowboarding while providing a fashion sense with interchangeable styles and/or colors.

### SUMMARY OF THE INVENTION

The present invention solves the problem of adjustably and comfortably fitting winter sports goggles to the head of the wearer while protecting the ears of the wearer with a wide, foldable headband for protecting against the cold. This combination of equipment replaces three pieces of equipment normally required to protect the winter athlete adequately, namely goggles with an elasticized strap, a headband, and ear protectors).

Attaching a one-piece headband to the goggles with a pair of plastic side clips, hook and loop fasteners (i.e. “Velcro”), or combinations of rivets and/or buttons, allows the headband to be fabricated from insulated material to provide protection against the cold. The material can be absorbent to soak up moisture and attractively colored or otherwise decorated with stitched, printed, attached designs and/or logos.

Providing a wide headband with material extending lower than the goggles creates a portion of the headband on each side of the wearer's head which completely covers each ear of the wearer for comfort and to protect against frostbite. In one embodiment, a generally U-shaped headband is made from an elongate piece of fabric disposed to be folded intermediate its longitudinal side edges over the band that otherwise secures a ski goggle to the wearer's head. Adjacent opposite ends, the folded headband has slightly enlarged portions disposed to cover the ears of the person wearing the goggle device. In one embodiment, each enlarged portion includes a pocket for holding an external heat source such as a warming cloth, chemical reaction material or small, self-contained battery-powered electronic heating unit.

### BRIEF DESCRIPTION OF THE DRAWINGS

Further features, objectives and advantages of the present invention will become clearer when referring to the following detailed description of preferred embodiments made with reference to the accompanying drawings in which:

FIG. 1 is a left side, plan view of one embodiment of this invention situated on the head of its wearer;

FIG. 2 is an outward, perspective view focusing on the ear covering component of this invention separated from the goggles component at both ends;

FIG. 3 is a top plan view of the ear covering exterior;

FIG. 4 is a top plan view of an intermediate layer of ear covering for the headband component with its side connectors removed for illustration purposes;

FIG. 5 is a partial cutaway, perspective view of the left end to the ear covering of FIGS. 3 and 4;

FIG. 6 is a right side, perspective view of another embodiment of this invention for retrofitting over an existing ski goggle/elastic band by installing the existing band about an ear covering component with its top lip flipped up; and

FIG. 7 is a right side, perspective view of the FIG. 6 ear covering component after its top lip has been flipped down about the elastic band and onto itself.

### DESCRIPTION OF PREFERRED EMBODIMENTS

Common features in the different views of this invention are shown with the same reference numeral(s). For alternate embodiments of the same component, there is consistent numbering though in the next hundred series. When referring to any numerical range herein, it should be noted that all numbers within the range, including every fraction or decimal between its stated minimum and maximum, are considered to be fully designated and disclosed hereby. As such, disclosing a total headband component length between about 17 and 20 inches would expressly cover all other lengths from 17.2, 17.4 and 18 inches up to and including about 19.5 and 19.75 inches.

The combination of sports gear in the figures include outdoor winter sports goggles shaped to the contours of the wearer's face and attached at both side edges to a strip of temperature-insulating fabric at least as wide as the side edge of the goggles and sufficiently long to extend fully about the wearer's head.

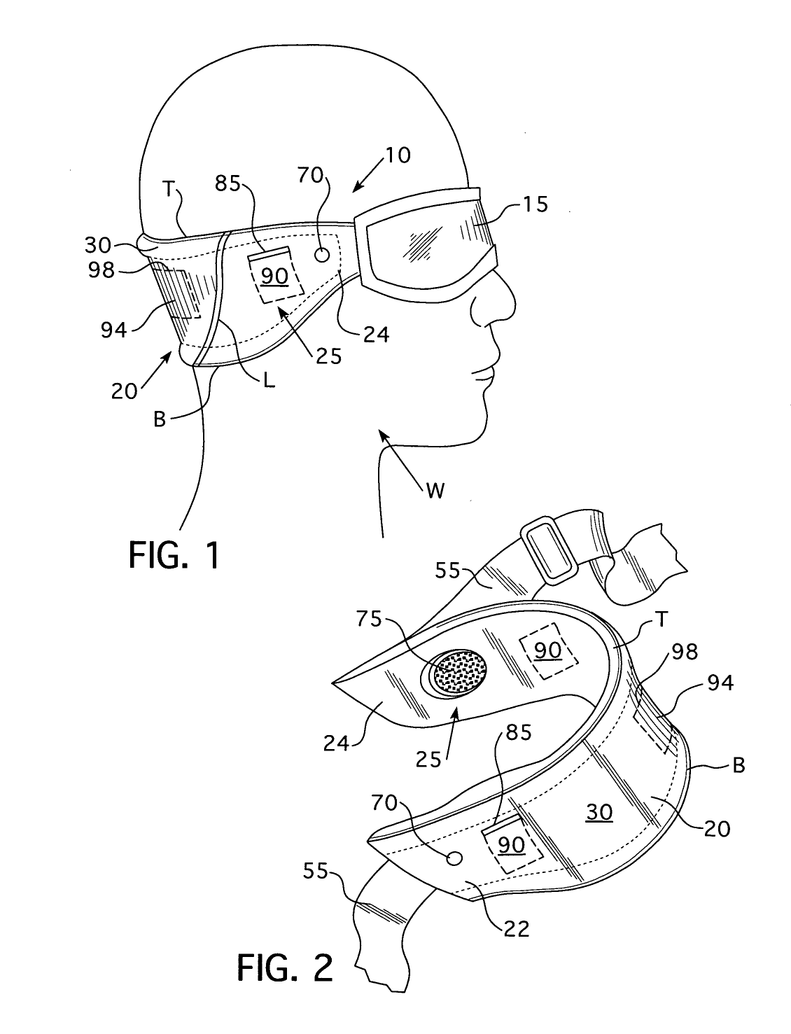

Extending downwardly from each strip of temperature-insulating fabric are ear-covering portions of fabric sufficiently wide to cover the wearer's ears. Together the goggles, strips of temperature-insulating fabric, and ear-covering portions create a novel combination that secures around the head of its wearer. An advertising logo, element “L” in FIG. 1, may be affixed to at least one strip of temperature-insulating material by printing or stitching or attaching a patch by gluing or stitching or other means.

One means of connecting insulating material to each side of the goggles comprises a strip of elasticized material threaded through a clip slot to an attaching clip. A pair of flexible hooks extending from the attaching clip are inserted in two openings in the side edge of the goggles, wherein the two openings on each side edge of the goggles are sufficiently large to receive the flexible hooks and the hooks snap into the openings to lock the headband to the goggles.

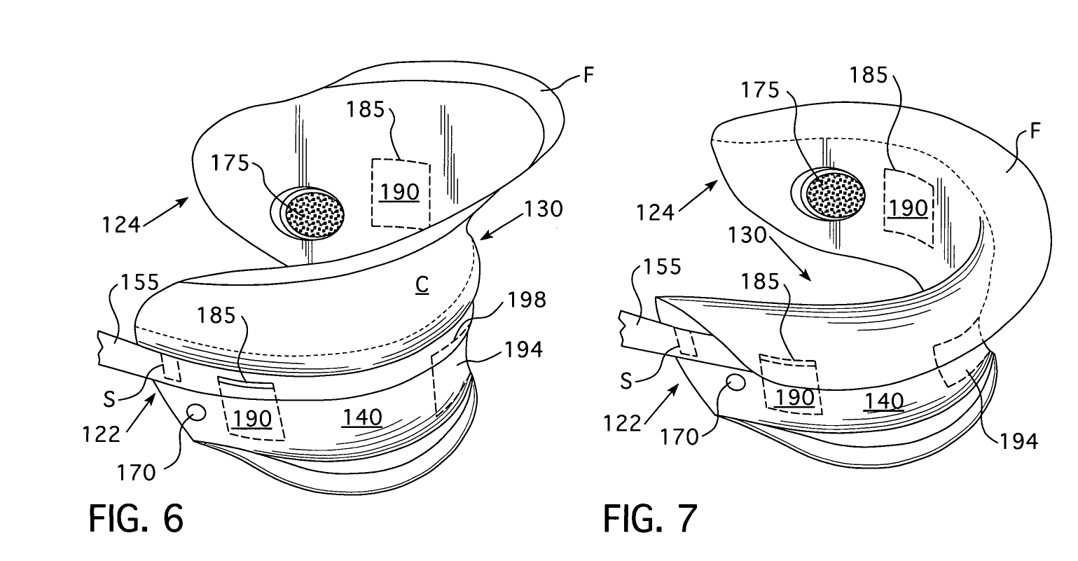

This invention also addresses retrofitting an existing ski goggle set. For such a system, already having its own thin elastic goggle strap, there is provided means for inserting the strap into a crease formed by folding over the temperature insulating fabric segment of a headband/ear warmer as per FIGS. 6 and 7. Preferably, at least one end of this elasticized material is permanently sewn to an end of the temperature-insulating fabric.

An alternate means of connecting each strip of temperature insulating material would provide an extension of the goggle frame to form a portion of the frame with a frame slot similar to the attaching clip molded to the frame without flexible hooks. The elasticized strip would then be threaded through that slot and attached to the temperature insulating material as above.

Typical goggles have a strip of foam (not shown) around an interior edge so that the foam strip engages with the wearer's face for a snug, comfortable fit. Many goggles further comprise at least one transparent screen for shielding wind and filtering ultraviolet rays. Ventilation openings along a top edge and a bottom edge of the goggles allow moisture to escape while a thin foam over such openings prevent wind from entering the goggles there through.

Preferably, the goggles are fabricated of molded plastic and the headband from one or more, doubled over sheets of fleece or other soft warm material having both insulating and absorption properties. Once connected altogether, this headband/ear covering/goggles combination fits over the head of its wearer by simply pulling back on the headband component before positioning: (i) the goggles over the wearer's eyes (or compact corrective lenses) and (ii) the ear covering portions over the wearer's ears.

The ear protection devices are preferably fabricated of a relatively warm fabric such as 100% knitted acrylic fabric. The fabric may be wool, cotton or blends thereof, including acrylic in the blends.

Each device includes near both ear ends a pocket or channel of sufficient size to hold an external, replaceable warming cloth. Alternately, one or both pockets can be used to hold packets of chemically inducing heat elements, electrical heaters (preferably, battery charging) or both. If not needed for warmth per se, one or both of these pockets can be used to hold a debit/credit card, ski pass, identification card/driver's license, hotel/chalet room key and/or small amounts of folded paper currency.

For greater utility, yet another pocket may be added to the central rear of this headband component for any or all of the aforementioned uses. While it is preferred that the heater pockets be accessed from the headband exterior, the other “central holder” pocket should only be accessed from inside that handband element. Furthermore, for ease of illustration, the heater pockets are shown with foldover flaps or press-sealed overlaps. They can also be fitted with zippers or other more permanent securing means as would be the preferred closure means for the centrally located, interior ID card/key holder pocket.

The width of the strap portions should be sufficient for folding onto itself along its longitudinal axis. To ensure securing of the folded portion to the remainder, one may employ cooperating, releasable fasteners, such as hook and fiber fasteners (e.g. Velcro), a plurality of snaps or buttons, or combinations of means. When folded over, the ear protecting elements better extend over the wearer's ears.

A single connector to the goggles is better than the two-piece straps of the prior art. This securing means provides a snug fit with elastic tensioning about the back of the wearer's head for staying put even when the wearer is subjected to wind force from movement such as in skiing, snowboarding and/or snowmobile riding. The ear protecting regions are preferably constructed from a soft flexible insulating material to prevent chafing of the wearer's ears. Suitable materials are acrylic, cotton, synthetics, or blends thereof.

Referring now to FIG. 1, there is shown a first embodiment of ear warming goggles, generally 10, as worn about the head and ears of its wearer W. This combination of winter headgear includes an eyewear protection, or goggle component 15, a main headband component 20 that connects at opposed ends 22 and 24 to goggle component 15 and ear covering/warming regions 25 within and integrally formed with headband component 20 on a preferred basis. In FIG. 1, only the right side of the head for wearer W is visible. It is to be understood, however, that the wearer's left side and left “half” of headband component 20 would resemble a mirror image of that shown in FIG. 1.

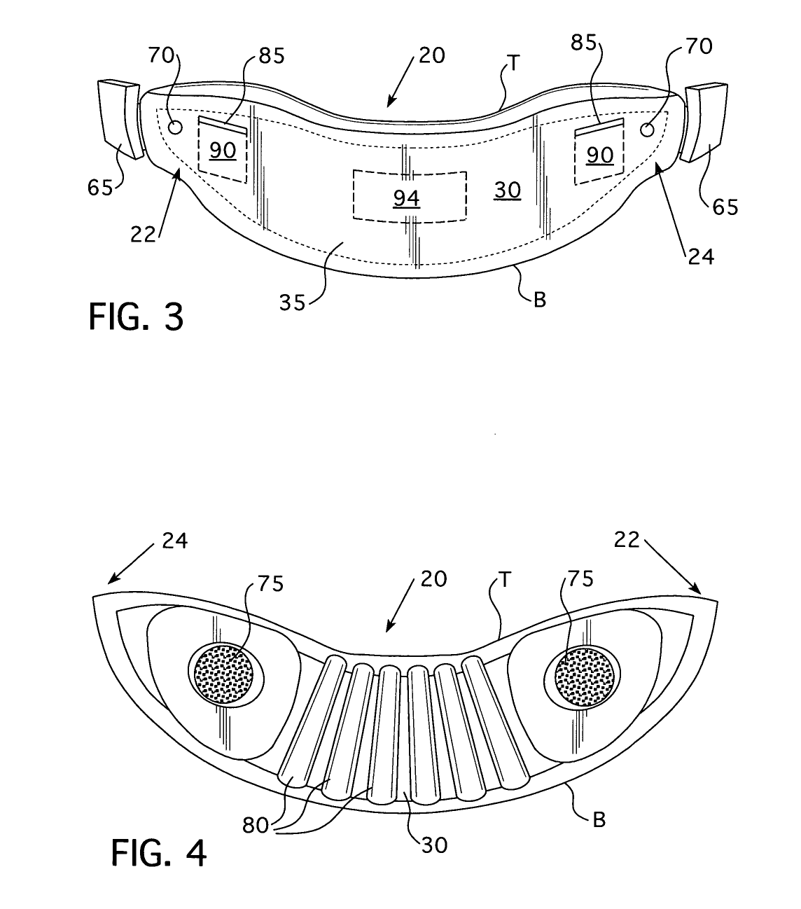

FIGS. 2-5 show other aspects of this first preferred embodiment in greater detail. Particularly, headband component 20 has a main body 30 sized to be at least as wide as the typical male skier's ear length, or substantially about 2.5 to 3 inches maximum. With an average adult human head circumference of about 23-25 inches, and a typical ski goggle width of about 6 to 8 inches, the overall length of main body 30 may run between about 17-19 or 20 total inches.

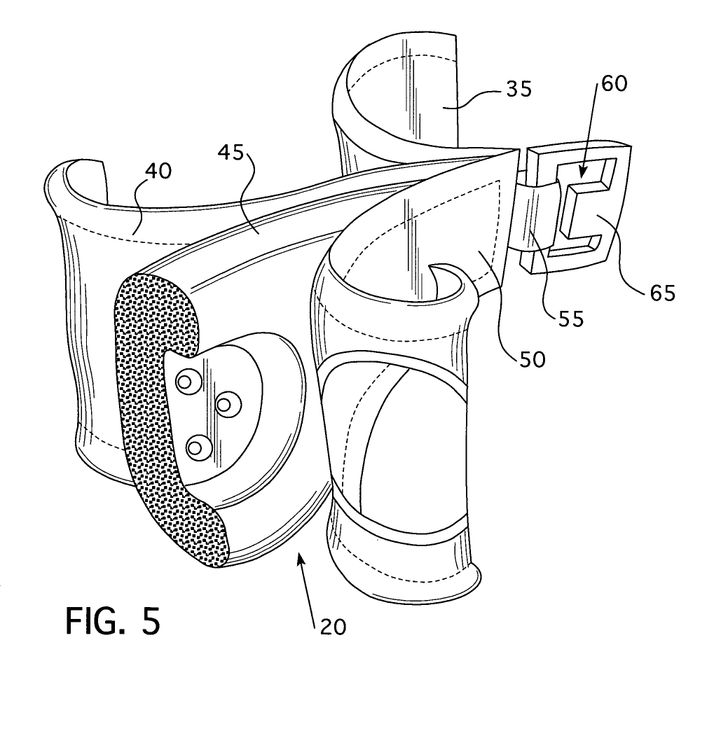

Ideally, main body 30 is comprised of multiple layers of material. From FIG. 5, for example, one embodiment was sectionally divided to better show: its outer, exterior design layer 35 through which heater pockets (not shown) could be accessed; an underlayer 40 adjacent to same; an intermediate “memory foam” layer 45 for better conforming to the wearer's ears (size and shape) for greater comfort, especially with prolonged wear/use; and a double sided, warm fleece interior layer 50.

To the far right in FIG. 5, there is also shown a section of elastic strap 55 that threads through a clip slot 60 in goggle attaching clip 65. One or more flexible hooks extend from attaching clip 65 for rapidly yet securely inserting into openings in the side edge of the goggles. When snapped or clicked into place, these hooks will lock the headband component 20 to the goggle component 15 of this one preferred embodiment.

As best seen in FIGS. 2 and 3, each opposed end 22, 24 of the exterior layer 35 to main body 30 has an ear hole 70. It connects to a much larger ear-contacting hole 75 through the main body's interior layer 50. For greater wearer comfort, longitudinal support strips 80 may extend substantially from the top T to bottom B of main body 30, as seen in accompanying FIG. 4.

A primary advantage of this winter sports goggle combination is that these goggles provide for external ear warming means, as needed. Particularly, the exterior layer 35 to main body 30 of headband component 20 has slits 85 at or close to the respective opposed ends 22, 24. These slits 85 enable wearer access to particular pockets 90 into which warming pads, chemical heating packets and/or electrical heating assemblies (all not shown) may be temporarily stored. For the latter device, battery powered assemblies are most preferred.

In addition to the preceding heater pockets 90, there is also preferably included a central storage pocket 94 accessed through the interior layer 50 of main body 30 through its own slotted entry, or slit 98. While not shown, it is understood that this slit 98 can be sealed or secured with zipper means, buttons, snaps, magnetic, Velcro (hook and loop tape), pressure seal strips or many other known or subsequently developed slot closure means. At a minimum, slit 98 may be covered with its own overlying flap (also not shown or numbered) for at least making sure that the pocket's securing means (zipper, strip or the like) does not cause any wearer discomfort by tangling in his/her hair.

FIGS. 6 and 7 sequentially depict a second embodiment for retrofitting a wearer's existing goggle and elastic strap with its own wraparound ear warming means. Particularly, in these two views, elastic strap 155 is first situated into a crease C in the main body 130 of headband component 120, said crease being formed by folding over a top lip to underlayer 140. The resultant fold or flap F keeps the elastic band from direct contact with the wearer's head. To better secure that strap 155 within the main body proper, at least one end may be stitched, or otherwise safely yet comfortably secured as indicated by stitch marks S in both FIGS. 6 and 7.

The foregoing description of the invention has been made with reference to several preferred embodiments. Persons skilled in the art will comprehend that minor changes and variations do not depart from the spirit and the scope of the invention defined by the following claims.

What is claimed is:

1. Ear warming goggles for winter sports comprises:
a goggle component shaped to fit against a wearer's face adjacent the wearer's eyes, said goggle component terminating in opposed side edges;
a headband component made from a temperature insulating fabric at least as wide as the side edges of the goggle component, said headband component being sufficiently long for extending around the wearer's head and having two downwardly extending panels for covering the wearer's ears; and
means for attaching opposed ends of the headband component to the side edges of the goggle component.

2. The ear warming goggles of claim 1 wherein each downwardly extending ear panel includes a pocketed region into which an external heat source may be temporarily stored.

3. The ear warming goggles of claim 2 wherein said pocketed region is manually sealable.

4. The ear warming goggles of claim 3 wherein said pocketed region is manually sealed with one or more means selected from: a zipper, button, snap, magnetic connector, Velcro (hook and loop tape) and a pressure seal strip.

5. The ear warming goggles of claim 2 wherein said pocketed region is covered with a removable flap of material.

6. The ear warming goggles of claim 2 wherein said pocketed region is accessed from an exterior of the headband component.

7. The ear warming goggles of claim 2 wherein said an external heat source is selected from the group consisting of: a patch that emits heat temporarily after chemicals thereon are mixed together or manually activated, and a battery-powered electric heater.

8. The ear warming goggles of claim 1, which further includes a large security pocket substantially central to the headband component for storing identification, credit cards, hotel keys, and currency.

9. The ear warming goggles of claim 8 wherein said large security pocket is only accessed from an interior of the headband component.

10. A combination winter goggle, headband and ear warmer comprises:
a goggle component shaped to fit against a wearer's face, said goggle component terminating in opposed side edges;
a headband component made from temperature insulating fabric at least as wide as the side edges of the goggle component, said headband component extending about the wearer's head and including downwardly extending panels for the wearer's ears, each panel including a pocketed region into which an external heat source may be temporarily stored;
means for attaching opposed ends of the headband component to the goggle component.

11. The goggle-headband-ear warmer combination of claim 10 wherein said pocketed regions may be manually sealed with one or more means selected from: a zipper, button, snap, magnetic connector, Velcro (hook and loop tape) and a pressure seal strip.

12. The goggle-headband-ear warmer combination of claim 10 wherein said pocketed regions are only accessed from an exterior of the headband component.

13. The goggle-headband-ear warmer combination of claim 10 wherein said external heat source is selected from the group consisting of: a patch that emits heat temporarily after chemicals thereon are mixed together or manually activated, and a battery-powered electric heater.

14. The goggle-headband-ear warmer combination of claim 10, which further includes a large security pocket substantially central to the headband component for storing identification, credit cards, hotel keys, and currency.

15. The goggle-headband-ear warmer combination of claim 14 wherein said large security pocket may only be accessed from an interior of the headband component.

16. A headband component for retrofitting winter goggles to add an ear warming component to same, said headband component comprising:
an elongate section of a temperature insulating fabric for connecting at opposed ends to opposed ends of the goggles and about the back of the goggle wearer's head, said elongate section having two downwardly extending panels for covering the goggle wearer's ears, each section including a pocketed region into which an external heat source may be temporarily stored.

17. The headband component of claim 16 wherein the section of temperature insulating fabric is adapted to wrap about any pre-existing elastic wear strap of the goggles.

18. The headband component of claim 16 wherein said pocketed regions are only accessed from an exterior of the section of temperature insulating fabric.

19. The headband component of claim 16 wherein said external heat source is selected from the group consisting of: a patch that emits heat temporarily after chemicals thereon are mixed together or manually activated, and a battery-powered electric heater.

20. The headband component of claim 16, which further includes a large security pocket substantially central to the headband component for storing identification, credit cards, hotel keys, and currency.
